(19) United States
(12) Patent Application Publication
(10) Pub. No.: US 2025/0250615 A1
(43) Pub. Date: Aug. 7, 2025

(54) Innovative Methods for Nucleic Acid Molecule Analysis, Gene Expression Profiling, In Situ Hybridization, and Comprehensive Molecule Interaction Studies Without Steps Such as Washing, qPCR, Probe-Coating, cDNA Generation and Incorporation of Fluorescence Into cDNA

(71) Applicant: Xiangfei Cheng, Montgomery Village, MD (US)

(72) Inventor: Xiangfei Cheng, Montgomery Village, MD (US)

(21) Appl. No.: 19/025,179

(22) Filed: Jan. 16, 2025

Related U.S. Application Data
(60) Provisional application No. 63/549,414, filed on Feb. 2, 2024.

(51) Int. Cl. C12Q 1/6818; C12Q 1/6841

(52) U.S. Cl. CPC C12Q 1/6818; C12Q 1/6841

(57) ABSTRACT

In my present innovation, the innovative methods introduce transformative advancements in analysis of nucleic acid molecules, gene expression profiling, in situ hybridization and molecular interaction studies, addressing key limitations of conventional methodologies. This versatile assay integrates miRNA, DNA, ctDNA, mRNA, siRNA, in situ hybridization, gene expression profiling, molecular interactions, and microarray technologies into efficient platform for precise molecular analysis. Key features of these innovative methods include the elimination of wash steps, a streamlined workflow, and enhanced sensitivity. The assay provides a qPCR-free solution to overcome size and amplification challenges in miRNA and siRNA assays and introduces a paradigm shift in genomic analysis. It enables in situ hybridization without relying on antibodies and radioactivity and delivers superior gene profiling without requiring wash steps, cDNA generation, or fluorescence incorporation into cDNA. These innovative features ensure heightened accuracy, reproducibility, and efficiency, establishing a new standard for molecular analysis.

### CROSS-REFERENCE TO RELATED APPLICATIONS

This application claims priority to Both U.S. Provisional Application No. 63/549,414 filed Feb. 2, 2024, and U.S. Provisional Application No. 63/549,819, filed Feb. 5, 2024 which are incorporated herein by reference.

### REFERENCE TO A SEQUENCE LISTING, A LARGE TABLE, OR A COMPUTER PROGRAM LISTING APPENDIX ON READ-ONLY OPTICAL DISC

Not Applicable

### STATEMENT REGARDING FEDERALLY SPONSORED RESEARCH OR DEVELOPMENT

Not Applicable

### BACKGROUND OF THE INVENTION

In the ever-evolving landscape of molecular biology and diagnostics, the demand for nucleic acid analysis, gene expression profiling, and molecules interaction assays, spanning miRNA, DNA, mRNA, siRNA, in situ hybridization, and microarray technologies, has consistently grown. However, conventional methodologies are often encumbered by challenges such as sensitivity, specificity, and efficiency, exacerbated by extensive wash steps and reliance on antibodies. Presenting my innovation, the groundbreaking assay —a innovative design poised to redefine the standards of precision, efficiency, and versatility in nucleic acid, gene expression profiling and molecules interaction analysis.

The traditional approach to miRNA and siRNA assays grapples with the elusive nature of these small RNA molecules, marked by low abundance and complex sequence homologies. The innovative methods strategically overcome these challenges by incorporating an innovative strategy that entirely eliminates the need for wash steps, preserving target integrity and ensuring maximal capture efficiency. Conventional amplification-based methods for detecting microRNA (miRNA) and small interfering RNA (siRNA) encounter constraints stemming from the diminutive sizes of these molecules. Additionally, challenges associated with amplification bias and artifacts further impede their effectiveness. The design of primers for qPCR is particularly challenging due to the compact sizes of miRNAs (19-25 nucleotides) and siRNAs (21-23 nucleotides). In my present innovation, I have overcome these challenges by eliminating the qPCR step from the detection process. This groundbreaking feature not only addresses the challenge and enhances sensitivity but also streamlines the assay workflow, making miRNA and siRNA analysis more accessible and eliminating the risk of target loss.

In the realm of DNA, mRNA, and siRNA assays, where heightened sensitivity and accuracy are crucial, the innovative methods lead this charge by introducing a paradigm shift—an inventive design that significantly eliminates wash steps without compromising specificity. This not only simplifies the assay process but also addresses challenges associated with low concentrations and cross-reactivity. The innovative methods herald a new era where precision in genomic, and transcriptomic is achieved with unprecedented efficiency, entirely avoiding the need for wash steps.

Traditional in situ hybridization methods often grapple with background signals and suboptimal penetration in complex tissues, demanding multiple wash steps that may compromise target retention. In my present innovation, the innovative methods tackle this issue head-on by optimizing probe characteristics and tissue processing steps, entirely eliminating the need for extensive washing after hybridization. This breakthrough allows for enhanced signal-to-noise ratios, providing researchers with unparalleled insights into spatial and temporal gene expression patterns without the risk of losing valuable targets. In conventional approaches, in situ hybridization methods rely on radioactivity or antibodies for target detection. Antibody-based methods face challenges in terms of specificity and efficiency, while radioactive methods pose potential safety risk. In my current invention, the innovative methods tackle this issue head-on by optimizing probe characteristics and tissue processing steps, entirely eliminating the need for radioactivity and antibodies in the in situ hybridization assay.

Unlike conventional microarray technologies, in my current invention, the innovative assay completely eliminates the need for a wash step, cDNA creation, and the incorporation of fluorophores into cDNA. The method introduces innovative features, ensuring that microarray analysis reaches new heights of accuracy and reproducibility without the requirement for cDNA generation, and wash steps.

In my present invention, a key advantage of these innovative methods lies in its ability to maintain target integrity without any wash steps, challenging the conventional belief that extensive washing is necessary for successful assays. Additionally, by eliminating the steps of qPCR and cDNA generation, my current invention represents a notable departure from conventional methods, providing a more direct and efficient route for nucleic acid analysis.

My present innovation, the innovative methods, represents a paradigm shift in molecular biology and offers advanced tools for diagnostics. By seamlessly integrating innovation with efficiency, these inventions not only address current challenges but also propels the field towards a future where precision meets simplicity, and the boundaries of what is possible are redefined.

### BRIEF SUMMARY OF THE INVENTION

In my present innovation, the innovative methods introduce a groundbreaking advancement in nucleic acid assays, in Situ Hybridization, gene expression profiling, and molecules interaction including but not limited to, nucleic acids, oligonucleotides, polynucleotides, small molecules, peptides and amino acids across various molecular biology applications. Overcoming challenges of conventional methods, such as sensitivity, specificity, and efficiency, these innovative methods cover a wide range of assays, including but not limited to miRNA, DNA, ctDNA, mRNA, siRNA, in situ hybridization, gene expression profiling, molecular interactions, and microarray technologies. It strategically eliminates the steps of qPCR, cDNA generation, and washing, preserving target integrity and ensuring maximal capture efficiency. This groundbreaking design enhances sensitivity, streamlines workflows, and eliminates the risk of target loss. Notably, the assay introduces unprecedented efficiency in genomic and transcriptomic, setting a new standard by avoiding the traditional requirement for wash steps. The innovative methods represent a paradigm shift, challenging conventional beliefs about extensive washing and reliance on antibodies, propelling molecular biology toward a future where precision meets simplicity, and redefining the boundaries of what is possible.

In my current innovation, these innovative methods represent a groundbreaking approach to gene expression profiling. Unlike traditional microarray methods, these innovative methods eliminate the need for cDNA generation from RNA and the incorporation of fluorescence into the cDNA. The innovative methods streamline the entire workflow, significantly reducing time consumption and eliminating potential inaccuracies associated with the cDNA generation process. This innovative approach enhances the efficiency and accuracy of gene expression profiling, marking a substantial advancement in the field.

In my present innovation, the innovative methods represent a groundbreaking advancement in nucleic acid concentration assays, gene expression profiling, in situ hybridization, and molecules interaction studies. Here are the key advantages of these innovative methods:
a) Elimination of Wash Steps: The innovative methods introduce a groundbreaking detection mechanism that entirely eliminates the need for wash steps. This innovative approach preserves target integrity and ensures maximal capture efficiency, addressing challenges associated with sensitivity and specificity.
b) Streamline Workflow and Enhance Sensitivity: The innovative methods entirely eliminate the wash step and strategically overcomes the elusive nature of small RNA molecules marked by low abundance and complex sequence homologies. The innovative methods enhance sensitivity and streamline workflow, making miRNA analysis more accessible and eliminating the risk of target loss.
c) Addressing Challenges in miRNA and siRNA Assays through a qPCR-Free Solution for Size and Amplification Issues: Traditional amplification-based methods for microRNA (miRNA) and siRNA detection encounter limitations arising from the small sizes of miRNAs and siRNAs, coupled with challenges related to amplification bias and artifacts. Designing primers for qPCR presents a significant obstacle due to the small size of miRNAs (19-25 nucleotides) and siRNAs (21-23 nucleotides). In my present innovation, the qPCR step is eliminated.
d) Paradigm Shift in Genomic Analysis: For miRNA, DNA, ctDNA, mRNA, and siRNA assays, the assay introduces a paradigm shift by entirely eliminating wash steps without compromising specificity. This design heralds a new era where precision in genomic and transcriptomic analysis is achieved with unprecedented efficiency.
e) In Situ Hybridization without Antibodies and Radioactivity: Traditional in situ hybridization methods often rely on radioactivity or antibodies for target detection. Antibody-based methods face challenges in specificity and efficiency, while the use of radioactivity introduces potential safety risks. My present innovative assay optimizes probe characteristics and tissue processing steps, entirely eliminating the need for antibodies and radioactivity in the in situ hybridization assay.
f) Elimination of cDNA Generation: The innovative methods eliminate the need for cDNA generation from RNA, streamlining a traditionally complex and error-prone step in gene expression profiling. This not only simplifies the process but also enhances result accuracy by avoiding potential variations introduced during cDNA synthesis.
g) Innovative Microarray Technology with Next-Generation Assay Methods: The present innovation introduces a groundbreaking assay that surpasses the limitations of microarray technologies, delivering unparalleled advancements in accuracy, reproducibility, and operational efficiency. Unlike conventional microarrays, this assay eliminates the need for wash steps, cDNA synthesis, and the incorporation of fluorescence into cDNA, thereby streamlining workflows and minimizing errors associated with these processes. By integrating innovative features and novel methodologies, this assay marks a significant leap forward, enabling faster, more reliable, and reproducible analysis across diverse applications.
h) Probe-Coating-Free: The assay's design eliminates the need for coating probes onto surfaces, simplifying the experimental setup. This enhances ease of use and eliminates potential variability associated with probe coating, ensuring more consistent and reproducible results across experiments.
i) Versatile Nucleic Acid Concentration Assays: The assay's applicability extends beyond gene expression profiling to nucleic acid concentration assays. Researchers can efficiently determine nucleic acid concentrations, making the assay versatile for various molecular biology applications.
j) Comprehensive Molecule Interaction Studies: Beyond nucleic acid applications, the innovative assay offers an advantage in quantitatively studying molecule interactions. The absence of washing and coating steps simplifies the process of analyzing molecule interactions, providing researchers with a powerful tool for elucidating complex biological mechanisms and studying small molecule synthesis and mechanisms.
k) Resource and Cost Efficiency: The simplified protocol, coupled with the elimination of qPCR, washing, and coating steps, contributes to resource and cost efficiency. Researchers can achieve robust results with fewer consumables, reducing experimental expenses and making the assay more accessible to a broader range of researchers and laboratories.
l) Rapid Turnaround: The combined advantages of a streamlined workflow, reduced steps, and no washing contribute to a rapid turnaround time for experiments. This is particularly valuable in time-sensitive research scenarios, enabling researchers to generate results quickly.

In my present innovations, the innovative methods can analyze diverse sample types, including plasma, serum, tissues, cultured cells, and challenging sample sources.

In summary, in my present innovation, the innovative methods mark a paradigm shift in molecular biology applications by eliminating the need for qPCR, cDNA generation, washing steps, and probe coating. Its advantages include elimination of washing, a simplified workflow, enhanced accuracy, versatility in applications, and resource efficiency, making it a powerful and efficient tool for nucleic acid concentration assays, gene expression profiling, in situ hybridization, and molecule interaction studies.

### BRIEF DESCRIPTION OF THE SEVERAL VIEWS OF THE DRAWING

FIG. 1. Principle of the Present Innovative Assays for Nucleic Acids and In Situ Hybridization. The probe is labeled with fluorophore and the complementary probe is labeled with quencher. After the labeled probe is hybridized to labeled complementary probe, the quencher plays important role of intramolecular quenching effect exerted by quencher on one fluorophore. a) The target to be assayed competes the hybridization of labeled probe to labeled complementary probe, resulting in enhancement of intensity of fluorescence. b) Labeled probe which are not hybridized to target are hybridized to labeled complementary probe, leading to intramolecular quenching effect exerted by quencher on one fluorophore, and elimination of washing. Intensity of fluorescence is proportional to the target concentration. The target concentration can be determined by measuring the intensity of fluorescence and calculating it according to the standard curve.

FIG. 2. Mechanism of Present Innovative Assays for Small Molecule or Peptide Interactions, and the Incorporation Of Molecules Or Functional Groups (Herein Referred To As The Donor Molecule) Into A Molecule (Herein Referred To As The Acceptor Molecule) During Molecular Synthesis. The probes are labeled with fluorophore, and target competitor are labeled with quencher. a) The target to be assayed competes the binding of labeled probe to the labeled target competitor, resulting in enhancement of intensity of fluorescence. b) Labeled probes which have not bound to target bind to labeled target competitor, leading to intramolecular quenching effect exerted by quencher on one fluorophore, and elimination of washing. Intensity of fluorescence is proportional to the target concentration. The molecule interaction or incorporation of donor molecule into an acceptor molecule during molecular synthesis is detected by the intensity of fluorescence, and the target concentration can be determined by measuring the intensity of fluorescence and calculating it according to the standard curve. The dissociation constant (Kd) can be calculated based on fluorescence measurements. The standard curve, generated using known concentrations of a standard substance, allows for the conversion of intensity of fluorescence to corresponding concentrations of the target analyte. In these innovative assays, the probe is referred as to a molecule or substance which are used to detect, bind to, or interact with the target to be studied. The target competitor is a molecule that competes with the target for binding to the probe or interacts with the probe.

FIG. 3. A schematic diagram of the invention for the concentration assays of nucleic acids including but not limited to miRNA, mRNA, siRNA, ctDNA and DNA, illustrating the step-by-step process and key components involved in the assay procedure.

FIG. 4. A schematic diagram of the invention for in situ hybridization, illustrating the step-by-step process and key components involved in the assay procedure.

FIG. 5. A schematic diagram of the invention for analysis of gene profiling, illustrating the step-by-step process and key components involved in the assay procedure. RNA isolation (exemplified with two samples) remains same as in the traditional approach.

FIG. 6. A schematic diagram of the invention for assay of dynamic molecular interactions involving small molecules, peptides, amino acids, nucleic acids, and oligonucleotide.

### DETAILED DESCRIPTION OF THE INVENTION

### Principles of Innovative Assays for Analysis of Nucleic Acids Within the Framework of My Current Innovation

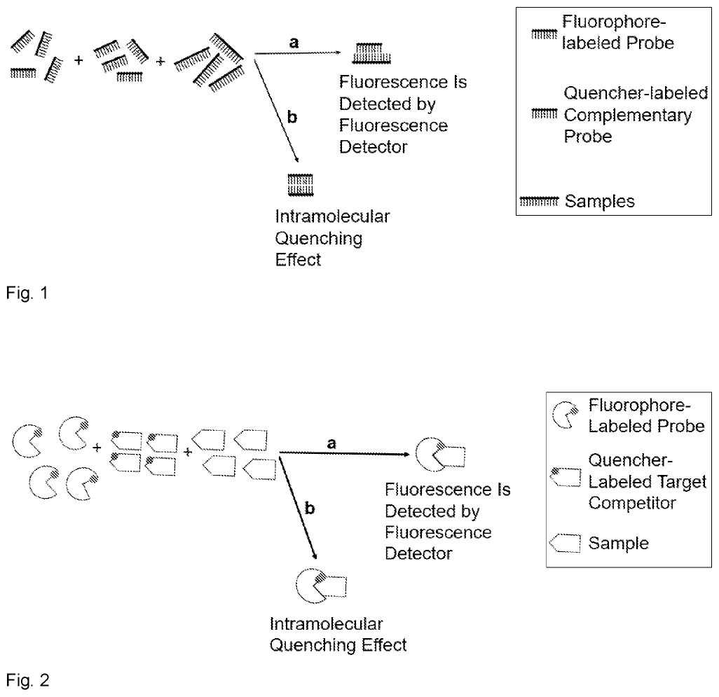

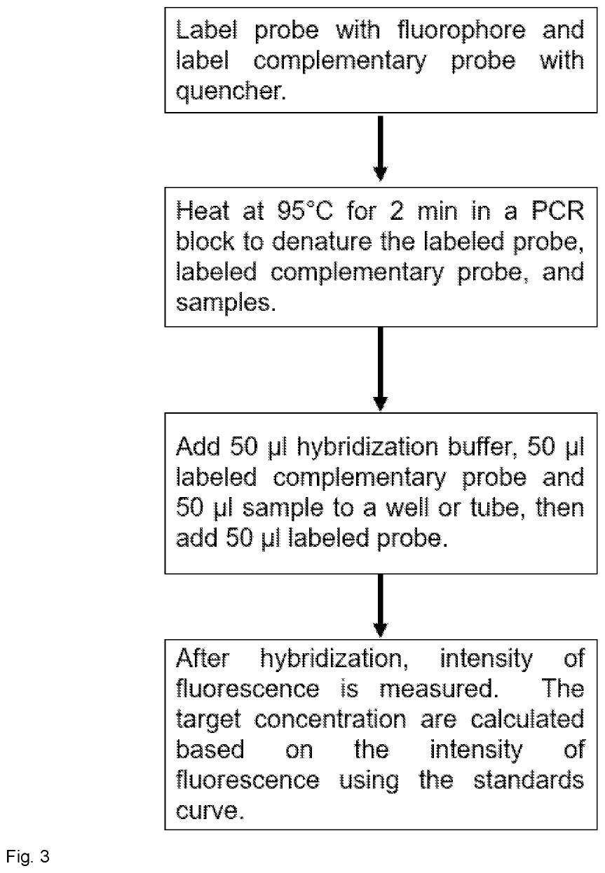

A Innovative Analysis Method Surpassing Conventional Concentration Assays of Nucleic Acids: My present innovative assays to measure concentration of nucleic acids including but not limited to miRNA, mRNA, siRNA and DNA surpass Conventional Concentration Assays (FIG. 3). The scientific rational (FIG. 1) behind the innovative assays is that the probe is labeled with fluorophore and the complementary probe is labeled with quencher. After labeled probe is hybridized to labeled complementary probe, the quencher plays important role of intramolecular quenching effect exerted by quencher on one fluorophore. The target to be assayed competes the hybridization of labeled probe to labeled complementary probe, enhancing intensity of fluorescence. Labeled probes which are not hybridized to target are hybridized to labeled complementary probe, leading to intramolecular quenching effect exerted by quencher on one fluorophore, and elimination of washing. Intensity of fluorescence is proportional to the target concentration. The target concentration can be determined by measuring the intensity of fluorescence and calculating it according to the standard curve. The standard curve, generated using known concentrations of a standard substance, allows for the conversion of intensity of fluorescence to corresponding concentrations of the target analyte. Alternatively, probe is labeled with quencher, and complementary probe with fluorophore.

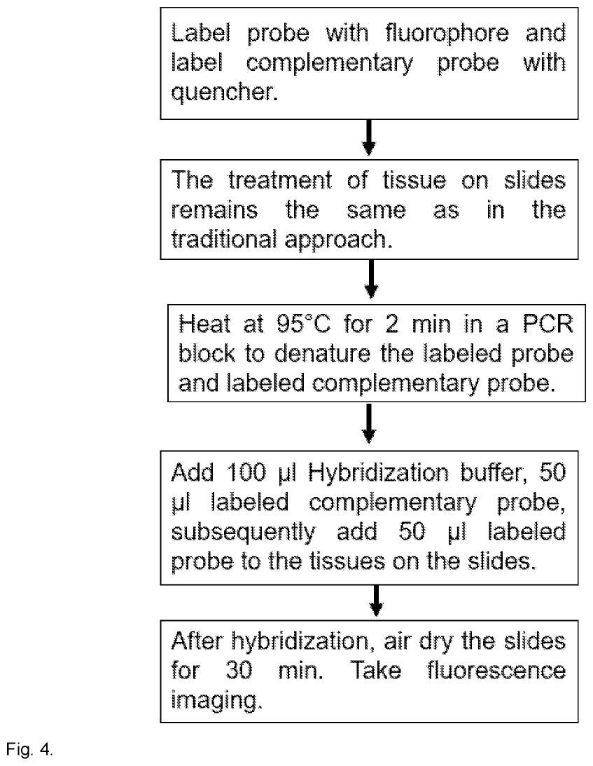

A Groundbreaking Analysis Method Surpassing Conventional In situ hybridization (FIG. 4): The scientific principle (FIG. 1) behind this innovation is that in situ hybridization assay, the probe is labeled with fluorophore and the complementary probe is labeled with quencher. The labeled probes hybridize to targets. The labeled probes which have not hybridized to targets hybridize to labeled complementary probe, leading to intramolecular quenching effect exerted by quencher on one fluorophore and elimination of washing. The hybridization of probe to target is detected by the fluorescence intensity.

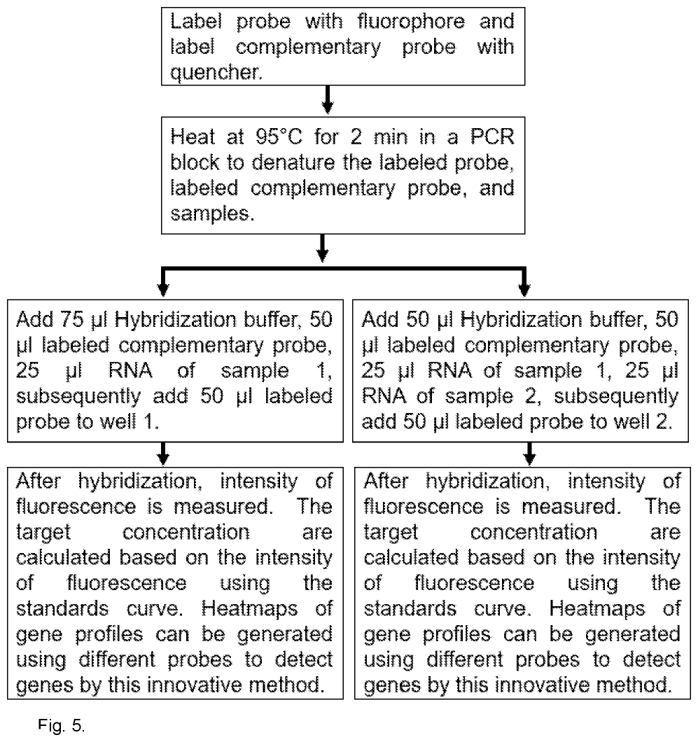

Gene Expression Profiling by This Innovative Method (FIG. 5): Underlying principle (FIG. 1) is that the probe is labeled with fluorophore and the complementary probe is labeled with quencher. After labeled probe is hybridized to labeled complementary probe, the quencher plays important role of intramolecular quenching effect exerted by quencher on one fluorophore. The target gene to be assayed competes the hybridization of labeled probe to labeled complementary probe. Labeled probes which are not hybridized to target are hybridized to labeled complementary probe, leading to intramolecular quenching effect exerted by quencher on one fluorophore, and elimination of washing. Intensity of fluorescence is proportional to the target concentration. The target concentration can be determined by measuring the intensity of fluorescence and calculating it according to the standard curve. The standard curve, generated using known concentrations of a standard substance, allows for the conversion of intensity of fluorescence to corresponding concentrations of the target analyte. Heatmaps of gene profiles can be generated using different probes to detect genes by this innovative method. Alternatively, probe is labeled with quencher, and complementary probe with fluorophore.

To illustrate the method for gene expression profiling, let's focus on a specific example—analyzing the genetic differences between normal cells (sample 1) and cancer cells (sample 2). The RNA isolation from cells, treatment of the probe, the complementary probe and samples (RNA) remain the same as in the conventional method. Hybridization buffer, labeled probe, labeled complementary probe and RNA of sample 1 are added to well 1 (microplate). Hybridization buffer, labeled probe, labeled complementary probe, RNA of sample 1 and RNA of sample 2 (amount ratio of RNA of sample 1 to RNA of sample 2 is 1:1) are added to well 2 (microplate). After Hybridization, fluorescence is measured. The target gene concentration is calculated based on the intensity of fluorescence using the standards curve.

Principles of the Innovative Assay for Analyzing Dynamic Molecular Interactions within the Framework of the Current Innovation

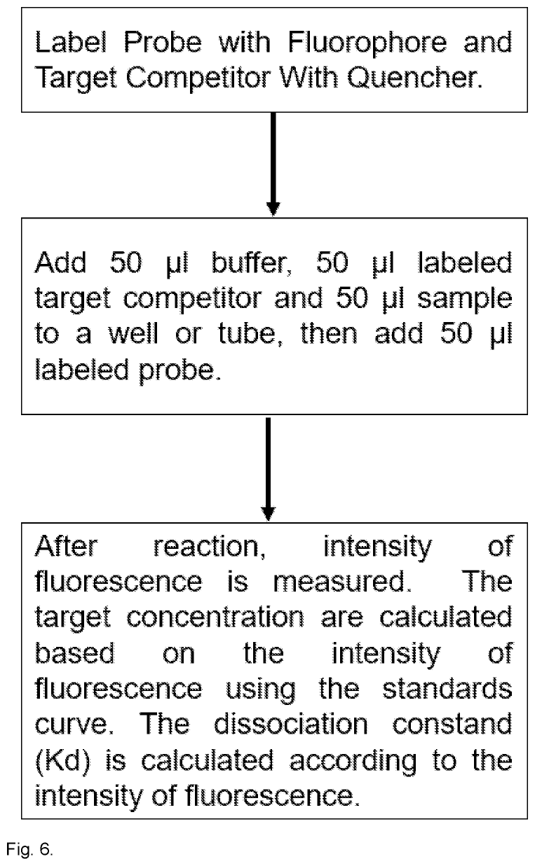

In my current innovation, the innovative assays are applied to investigate dynamic molecular interactions including detection of the incorporation of molecules or functional groups (herein referred to as the donor molecule) into a molecule (herein referred to as the acceptor molecule) during molecular synthesis (FIG. 6). These assays cover a broad spectrum, encompassing, but not limited to, small molecules, amino acids, peptides, nucleic acids, oligonucleotides and polynucleotides. These assays are applied to explore interactions between any combination of these molecules, providing a versatile platform for comprehensive molecular studies. In current invention, a probe is referred as to a molecule or substance (herein referred to as the acceptor molecule) which are used to detect, bind to, or interact with the target (herein referred to as the donor molecule) to be studied. A target competitor is a molecule that competes with the target for binding to the probe or interacts with the probe. The conceptual framework (FIG. 2) is that the probes are labeled with fluorophore, and target competitor are labeled with quencher. The target to be assayed competes the binding of labeled probe to the labeled target competitor, resulting in enhancement of intensity of fluorescence. Labeled probes which have not bound to target bind to labeled target competitor, leading to intramolecular quenching effect exerted by quencher on one fluorophore, and elimination of washing. Intensity of fluorescence is proportional to the target concentration. The molecule interaction or incorporation of donor molecule into an acceptor molecule during molecular synthesis is detected by the intensity of fluorescence, and the target concentration can be determined by measuring the intensity of fluorescence and calculating it according to the standard curve. The dissociation constant (Kd) can be calculated based on fluorescence measurements. The standard curve, generated using known concentrations of a standard substance, allows for the conversion of intensity of fluorescence to corresponding concentrations of the target analyte.

Alternatively, probe can be labeled with quencher, and target competitor with fluorophore. If the molecule is a nucleic acid, the labeling and the treatment procedures align with those outlined in the “Synthesize Probe and Complementary probe, and Label Both Molecules (method), and Innovative Concentration Assay of Nucleic Acids Including but Not Limited to miRNA, mRNA, siRNA, ctDNA and DNA.”

Importantly, it is crucial to note that the labeling must not impact probe interaction with target competitor and target, and it should ensure that quencher can efficiently quenches the fluorophore when the labeled probe interacts with the labeled target competitor. The decision on selection of the pair of fluorophore and quencher can be determined through a screening test.

### Method

Synthesize Probe and Complementary probe, and Label Both Molecules: In my present invention, for genetic materials assays including but not limited to miRNA, mRNA, siRNA, ctDNA and DNA assays, as well as gene expression profiling and in situ hybridization, a probe is a short strand of oligonucleotide that is complementary to a specific sequence of nucleotides in a target DNA or RNA molecule. In my present innovation, Dns or its derivatives serves as an example of a fluorophore, Dnp or Dbc or their derivatives serves as an example of a quencher. The probes are labeled with fluorophore, and complementary probe are labeled with quencher:
a) Label Probe with Fluorophore: Select the probe sequence which is complementary to the target to be assayed. The incorporation of Fluorophore into probes is achieved, including but not limited to, during the probe's synthesis process. Alternatively, all nucleotides to be synthesized for the probe are labeled with fluorophore. The probes are synthesized using the nucleotides which have been labeled with the fluorophore, or after the probes are synthesized with unlabeled nucleotides, the probes are labeled with fluorophore.
b) Label Complementary probe with quencher: Select sequence of complementary probe which is complementary to the probe. The incorporation of quencher into complementary probe is achieved, including but not limited to, during the complementary probe synthesis process. Alternatively, all nucleotides to be synthesized for the complementary probe are labeled with quencher. The complementary probes are synthesized using the nucleotides which have been labeled with quencher, or after the complementary probe are synthesized with unlabeled nucleotides, the complementary probe are labeled with quencher.

Alternatively, probe can be labeled with quencher, and complementary probe with fluorophore except for in situ hybridization. In my present innovation, for in situ hybridization assay, the probe is labeled with fluorophore and the complementary probe is labeled with quencher.

Both fluorophore and quencher can label either all nucleotides or a specific subset of nucleotides. Importantly, it is crucial to note that the labeling must not impact the hybridization of probe to complementary probe, and it should ensure that quencher can efficiently quenches the fluorescence after the labeled probes are hybridized to the labeled complementary probe, irrespective of whether all nucleotides are labeled, only a partial set is labeled, or different labeling and creation methods are used for the generation of the labeled probe and labeled complementary probe. The decision to label all nucleotides or only a subset, method to be used to generate the labeled probe and labeled complementary probe can be determined through a screening test.

Concentration Assay of Nucleic Acids Including but not limited to miRNA, mRNA, siRNA ctDNA and DNA: Heat at 95° C. for 2 min in a PCR block to denature the labeled probe, labeled complementary probe, and samples. Add 50 μl hybridization buffer, 50 μl labeled complementary probe and 50 μl sample to a well of microplate or tube, then add 50 μl labeled probe. Molar ratio of labeled probe to labeled complementary must be 1:1. After hybridization, intensity of fluorescence is measured. The target concentration is calculated based on the intensity of fluorescence using the standards curve.

Gene Expression Profiling by This Innovative Method: To illustrate the method, let's focus on a specific example—analyzing the genetic differences between normal cells (sample 1) and cancer cells (sample 2). RNA isolation and treatment remain the same as in the traditional approach. For this analysis, the procedures are as follows (using a microplate as an example):
a) Adjust the concentration of RNA in sample 1 to match that of RNA in sample 2; b) Heat at 95° C. for 2 min in a PCR block to denature the labeled probe, labeled complementary probe, and samples;
c) Add 75 μl Hybridization buffer, 50 μl labeled complementary probe, and 25 μl RNA of sample 1 to well 1. Add 50 μl Hybridization buffer, 50 μl labeled complementary probe, 25 μl RNA of sample 1, 25 μl RNA of sample 2 to well 2;
d) Add 50 μl labeled probe to well 1 and well 2, respectively;
e) After hybridization, fluorescence is measured. The target gene concentration is calculated based on the intensity of fluorescence using the standards curve.

Heatmaps of gene profiles can be generated using different probes to detect genes by this innovative method.

It is important to note that in all the assay, the molar ratio of labeled probe to labeled complementary probe must be 1:1. The target concentration can be determined by measuring the intensity of fluorescence and calculating it according to the standard curve. The standard curve, generated using known concentrations of a standard substance, allows for the conversion of intensity of fluorescence to corresponding concentrations of the target analyte.

Innovative In Situ Hybridization: In my current innovation, in situ hybridization, the procedures for treatment of tissue on slides, including deparaffinization, antigen retrieval, slide dehydration and denature before hybridization, remain the same as in the traditional approach. Heat at 95° C. for 2 min in a PCR block to denature the labeled probe and labeled complementary probe. Add 100 μl Hybridization buffer, 50 μl labeled complementary probe, subsequently add 50 μl labeled probe to the sample on the slide. After hybridization, air dry the slides for 30 min. Take a fluorescence imaging.

Assay of Molecule Interaction including detection of incorporation of molecules or functional groups into a molecule during molecular synthesis: Add 50 μl buffer, 50 μl labeled target competitor and 50 μl sample to a well of microplate or tube, then add 50 μl labeled probe. After reaction, fluorescence is measured. Molecule interaction or incorporation of molecules or functional groups into a molecule is detected by measuring intensity of fluorescence. The target concentration is calculated based on the intensity of fluorescence using the standards curve. The target concentration can be determined by measuring the intensity of fluorescence and calculating it according to the standard curve. The dissociation constant (Kd) can be calculated based on fluorescence intensity. The standard curve, generated using known concentrations of a standard substance, allows for the conversion of intensity of fluorescence to corresponding concentrations of the target analyte. In all the assay, the molar ratio of labeled probe to labeled target competitor must be 1:1.

In my current innovation, users have the flexibility to choose between microplates, tubes, or chips. The volumes of samples, buffers, and all reagents are adjustable and determined through a screening test, with the exception of specific ratios. The handling of samples, reagents, and buffer care remains consistent with traditional methods.

In my current innovation, the fundamental principle of the innovative methods relies on intramolecular quenching effect exerted by quencher on one fluorophore (1, 2). In certain assays, the pair of variables, fluorophore and quencher, is interchangeable with the pair of fluorophore and enhancer. In certain embodiments, the pair of variables, fluorophore and quencher can be replaced with the pair of detection agents and the impactor which can play role of intramolecular effect on the detection agents' signal.

### DISCUSSION

My current invention represents a significant advancement in the field of nucleic acid analysis, including nucleic acid concentration assays, gene expression profiling, in situ hybridization, and molecular interaction studies. It offers a comprehensive solution to several challenges associated with traditional methodologies. The discussion below highlights key aspects of the invention and its potential impact on research, diagnostics, and other applications.

Streamline Assay Workflow: The elimination of probe-coating, a common bottleneck in nucleic acid assays, streamlines the overall workflow. Traditional methods often involve intricate steps for surface modification, leading to increased assay duration and cost. My present innovative assays not only reduce assay time but also simplify experimental protocols, making them more accessible to researchers with varying levels of expertise.

Speed and Cost Efficiency: The rapid analysis provided by the inventive assays is a notable advantage, particularly in time-sensitive experiments and high-throughput applications. The reduction in overall assay time translates to increased experimental throughput and quicker data acquisition. Furthermore, the inherent cost savings resulting from the elimination of probe-coating contribute to the economic feasibility of the technology for both research and diagnostic purposes.

Versatility in Application: The versatility of the assays, spanning from gene expression profiling to in situ hybridization, and molecule interaction studies, expands the range of applications for which the technology can be employed. This adaptability is crucial for researchers exploring various aspects of molecular biology, genomics, and diagnostics, allowing them to tailor the assays to meet specific experimental needs.

Addressing Challenges in miRNA and siRNA Assays with a qPCR-Free Solution for Size and Amplification Issues: Traditional amplification-based methods for microRNA (miRNA) and small interfering RNA (siRNA) detection face constraints due to the diminutive sizes of miRNAs and siRNAs, along with challenges associated with amplification bias and artifacts. Designing primers for qPCR poses a substantial obstacle given the small size of miRNAs (19-25 nucleotides) and siRNAs (21-23 nucleotides). In this innovative approach, the qPCR step is entirely eliminated.

Simplifies Workflow for Microarray: Traditional microarray methodologies necessitate a multi-step process involving cDNA generation and fluorescence incorporation. The present innovation eliminates the need for the often intricate and time-consuming procedure associated with traditional microarray methodologies. The elimination of these steps not only expedites the entire microarray workflow but also significantly reduces the complexity of experimental procedures.

Direct Analysis Without Washing Steps: The exclusion of washing steps in the assay protocol is another notable feature of this innovation. Traditional protocols often involve meticulous washing procedures to remove unbound or excess molecules, adding complexity to the experimental workflow. The absence of washing steps not only accelerates the analysis but also simplifies the procedure, making it more accessible to a broader range of researchers.

### IN CONCLUSION

The presented innovative methods mark a paradigm shift in molecular analysis, offering an integrated solution for nucleic acid concentration assays, gene expression profiling, in situ hybridization, and comprehensive molecule interaction studies. With the elimination of qPCR, cDNA generation, probe-coating, and washing steps, our innovative approach not only streamlines the experimental workflow but also enhances accuracy and efficiency.

These innovative methods' adaptability for analysis of diverse nucleic acid types, including but not limited to miRNA, siRNA, mRNA, ctDNA and DNA, showcases its versatility. The elimination of time-consuming steps, such as cDNA generation and washing, not only reduces experimental time but also minimizes the risk of errors and sample contamination.

Furthermore, my innovative design facilitates comprehensive molecule interaction studies. The absence of probe-coating steps ensures a simplified experimental setup and consistent, reproducible results across experiments.

My present innovative methods promise to be a valuable tool for researchers, offering robust molecular analysis with reduced consumable requirements. The rapid turnaround time for experiments is particularly advantageous in time-sensitive research scenarios, enabling researchers to expedite their studies.

In conclusion, the presented innovative methods represent a groundbreaking advancement in molecular biology, providing a powerful and versatile platform for a wide range of applications. Its streamlined workflow, elimination of washing, cDNA generation, and adaptability make it a promising innovation with the potential to significantly impact the field of molecular analysis.

1: A method for determining nucleic acid concentration, the method comprising:
a) Label probe with fluorophore and label complementary probe with quencher;
b) Denature the labeled probe, labeled complementary probe, and samples;
c) Add the labeled complementary probe, sample and hybridization buffer to a well or tube, then add labeled probe for hybridization competition (Molar ratio of labeled probe to labeled complementary probe must be 1:1);
d) After hybridization, intensity of fluorescence is measured. The target concentration is calculated based on the intensity of fluorescence using the standards curve.

2: A method for gene profiling, the method comprising:
a) Label probe with fluorophore and label complementary probe with quencher;
b) Isolate RNA from sample 1 and sample 2, respectively (exemplified with two samples). Adjust the concentration of RNA in sample 1 to match that of RNA in sample 2;
c) Denature the labeled probe, labeled complementary probe, and the RNA samples;
d) Add Labeled complementary probe, RNA from sample 1 and hybridization buffer to well 1; Add Labeled complementary probe, RNA from Sample 1, RNA from Sample 2 and hybridization buffer to well 2;
e) Add labeled probe to well 1 and well 2, respectively;
f) After hybridization, fluorescence is measured. The target gene concentration is calculated based on the intensity of fluorescence using the standards curve. Heatmaps of gene profiles can be generated using different probes to detect genes by this innovative method.

3: A method for in situ hybridization, the method comprising:
a) Label probe with fluorophore and label complementary probe with quencher;
b) The treatment of tissue on slides, including deparaffinization, antigen retrieval, slide dehydration and denature prior to hybridization, remains the same as in the traditional approach;
c) Denature the labeled probe and labeled complementary probe;
d) Add hybridization buffer and labeled complementary probe to the sample on the slide, then add labeled probe;
e) After hybridization, air dry the slides for 30 min. Take a fluorescence imaging.

4: A method for Assay of Molecule Interaction including detection of the incorporation of molecules or functional groups into a molecule during molecular synthesis, the method comprising:
a) Label probe with fluorophore and label target competitor with quencher;
b) Add labeled target competitor, sample and the buffer to a well, then add labeled probe;
c) After reaction, fluorescence is measured;
d) Molecule interaction or incorporation of molecules or functional groups into a molecule is detected by measuring intensity of fluorescence. The target concentration is calculated based on the intensity of fluorescence using the standards curve. The molecule interaction or incorporation of donor molecule into an acceptor molecule during molecular synthesis is detected by the intensity of fluorescence, and the target concentration can be determined by measuring the intensity of fluorescence and calculating it according to the standard curve. The dissociation constant (Kd) can be calculated based on fluorescence intensity. The standard curve, generated using known concentrations of a standard substance, allows for the conversion of intensity of fluorescence to corresponding concentrations of the target analyte. In all the assay, the molar ratio of labeled probe to labeled target competitor must be 1:1.

5: The design strategy of the innovative methods of claims 1-3, wherein the probe is labeled with fluorophore and the complementary probe is labeled with quencher. After labeled probe is hybridized to labeled complementary probe, the quencher plays important role of intramolecular quenching effect exerted by quencher on one fluorophore. The target to be assayed competes the hybridization of labeled probe to labeled complementary probe. Labeled probes which are not hybridized to target are hybridized to labeled complementary probe, leading to intramolecular quenching effect exerted by quencher on one fluorophore, and elimination of washing.

6: The design strategy of the innovative method of claim 4, wherein the probes are labeled with fluorophore, and target competitor are labeled with quencher. The target to be assayed competes the binding of labeled probe to the labeled target competitor. Labeled probes which have not bound to target bind to labeled target competitor, leading to intramolecular quenching effect exerted by quencher on one fluorophore, and elimination of washing

7: The methods of claim 1-4, wherein the pair of variables, fluorophore and quencher, is interchangeable with the pair of fluorophore and enhancer. In certain embodiments, the pair of variables, fluorophore and quencher can be replaced with the pair of detection agents and the impactor which can play role of intramolecular effect on the detection agents' signal.
